(19) United States
(12) Patent Application Publication
(10) Pub. No.: US 2026/0078332 A1
(43) Pub. Date: Mar. 19, 2026

(54) DRUG SCREENING PLATFORM FOR TUMOR MICROENVIRONMENT SIMULATION

(71) Applicant: National Tsing Hua University, Hsinchu City (TW)

(72) Inventors: Zih-Yin CHAO, Hsinchu City (TW); Cheng-Hsien LIU, Hsinchu City (TW)

(21) Appl. No.: 18/913,204

(22) Filed: Oct. 11, 2024

(30) Foreign Application Priority Data
Sep. 19, 2024 (TW) 113135480

(51) Int. Cl. C12M 1/34; B01L 3/00; C12M 1/00; C12M 3/06

(52) U.S. Cl. CPC C12M 41/46; B01L 3/502715; C12M 23/16; C12M 23/34; C12M 29/06; C12M 41/12; B01L 2200/16; B01L 2300/0883

(57) ABSTRACT

A drug screening platform for tumor microenvironment simulation includes a tumor microenvironment simulating chip, a drug testing chip, and a permeable thin film disposed between the tumor microenvironment simulating chip and the drug testing chip. The tumor microenvironment simulating chip includes a microenvironment building unit which is used for conducting a chemical reaction to generate an acid gas, an alkaline gas and/or an oxygen concentration for simulating the tumor microenvironment. The drug testing chip includes a testing unit which is used for allowing interaction of a therapeutic drug with a cell-containing hydrogel. An orthographic projection of the testing unit falls on the microenvironment building unit. The permeable thin film is used for diffusing the acid gas, the alkaline gas and/or the oxygen concentration generated by the microenvironment building unit to the testing unit.

### CROSS-REFERENCE TO RELATED APPLICATION

This application claims priority to Taiwanese Invention Patent Application No. 113135480, filed on, September, 19, 2024, the entire disclosure of which is incorporated by reference herein.

### FIELD

The disclosure relates to a drug screening platform, and more particularly to a drug screening platform for tumor microenvironment simulation.

### BACKGROUND

The effectiveness of cancer treatment is closely related to tumor microenvironment. As a result, if a drug screening platform that is used for screening a therapeutic cancer drug includes the function of simulating a tumor growing microenvironment, precise and accurate testing results may be obtained. This type of drug screening platform may be, for example, a drug screening platform that simulates a hyperthermic intraperitoneal chemotherapy that was disclosed in Taiwanese Invention patent no. TW I795812B. The drug screening platform that simulates the hyperthermic intraperitoneal chemotherapy, through a dielectrophoresis force, arranges cells into a three dimensional structure so as to simulate a three dimensional tumor microenvironment.

### SUMMARY

Therefore, an object of the present disclosure is to provide a drug screening platform that is provided with a novel approach to simulate a tumor microenvironment.

Thus, the drug screening platform for tumor microenvironment simulation in the present disclosure includes a tumor microenvironment simulating chip, a drug testing chip, and a permeable thin film disposed between the tumor microenvironment simulating chip and the drug testing chip.

The tumor microenvironment simulating chip includes a microenvironment building unit. The microenvironment building unit is used for conducting a chemical reaction to generate an acid gas, an alkaline gas and/or an oxygen concentration for simulating the tumor microenvironment.

The drug testing chip includes a testing unit. The testing unit is used for allowing interaction of a therapeutic drug with a cell-containing hydrogel. An orthographic projection of the testing unit falls on the microenvironment building unit.

The permeable thin film is disposed between the tumor microenvironment simulation chip and the drug testing chip. The permeable thin film is used for diffusing the acid gas, the alkaline gas and/or the oxygen concentration generated by the microenvironment building unit to the testing unit.

### BRIEF DESCRIPTION OF THE DRAWINGS

Other features and advantages of this invention will be clearly presented in the embodying manners with reference to the drawings, in which:

FIG. 1 is a schematic top view of an embodiment of a drug screening platform for tumor microenvironment simulation according to the present disclosure;

FIG. 2 is a schematic side view of the embodiment;

FIG. 3 is a top view of a tumor microenvironment simulating chip of the embodiment; and

FIG. 4 is a top view of a drug testing chip of the embodiment.

### DETAILED DESCRIPTION

Before the present invention is described in detail, it should be noted that similar elements are represented by the same reference numeral in the following description.

The present disclosure will be further explained by the following embodiment(s). However, it should be understood that the embodiment(s) is (are) for illustrative purposes only and should not be interpreted as a limitation for the present disclosure.

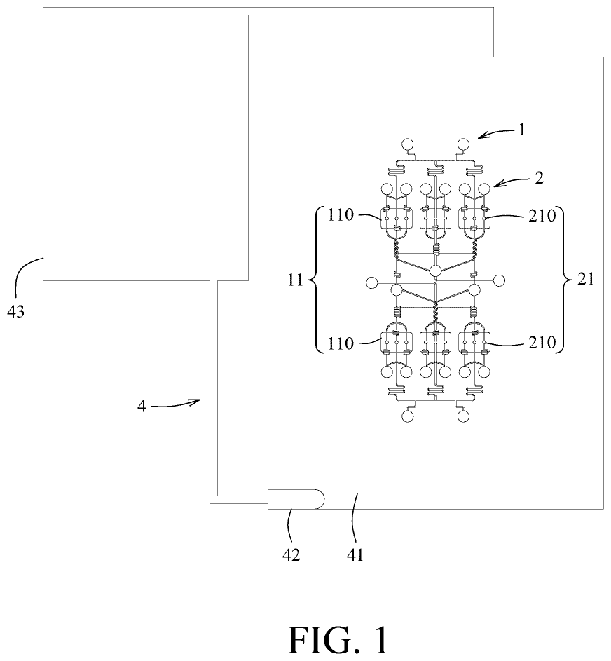

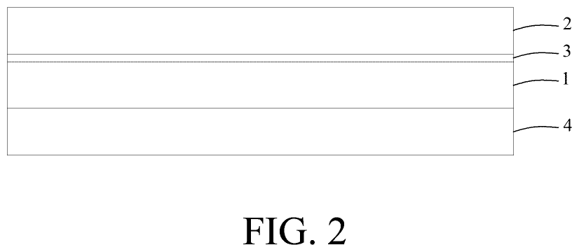

Referring to FIG. 1 and FIG. 2, an embodiment of a drug screening platform for tumor microenvironment simulation of the present disclosure includes a tumor microenvironment simulation chip 1, a drug testing chip 2, a permeable thin film 3 (not depicted in FIG. 1), and a heating apparatus 4.

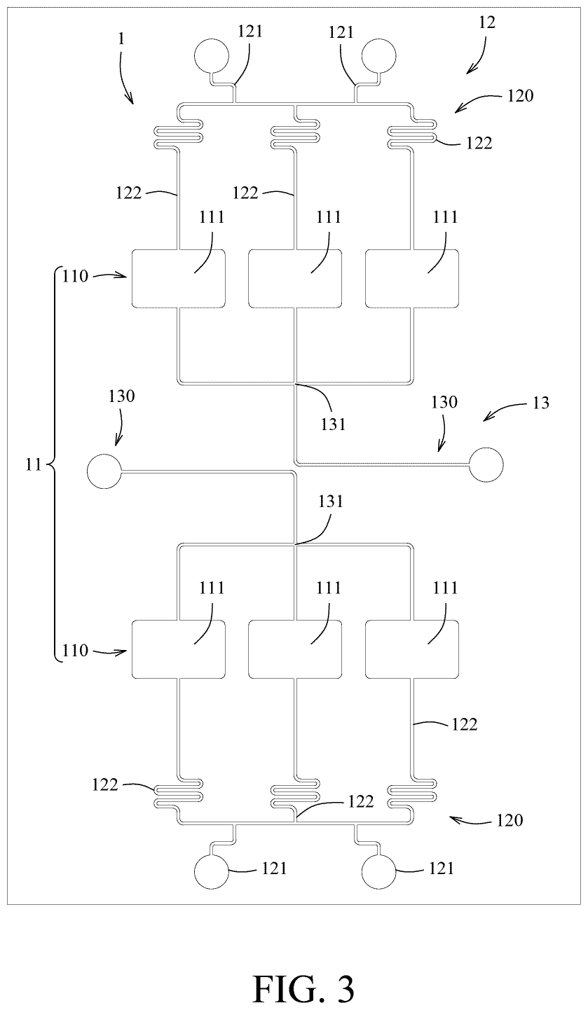

Referring to FIG. 1 and FIG. 3, the tumor microenvironment simulating chip 1 includes a microenvironment building unit 11, an input unit 12 (not depicted in FIG. 1) and an output unit 13 (not depicted in FIG. 1), which are in fluid communication with the microenvironment building unit 11.

According to the present disclosure, the microenvironment building unit 11 is used for conducting a chemical reaction to generate an acid gas, an alkaline gas and/or an oxygen concentration for simulating a tumor microenvironment. The microenvironment building unit 11 has two chemical reaction regions 110 which are disposed in opposing directions from each other. Each of the chemical reaction regions 110 has a plurality of reaction chambers 111 which are arranged spaced apart from one another. The chemical reaction is conducted in the plurality of reaction chambers 111. The number of the reaction chambers 111 may be flexibly adjusted according to actual requirements. In the present embodiment, each of the chemical reaction regions 110 has three reaction chambers 111.

According to the present disclosure, the input unit 12 is used for delivering chemicals that conduct the chemical reaction into the microenvironment building unit 11. The input unit 12 includes two input channels 120 which are disposed in opposing directions from each other and each of which is in fluid communication with a respective one of the chemical reaction regions 110. Each of the input channels 120 includes a plurality of injection sections 121 and a plurality of distributary sections 122 which fluidly communicate the injection sections 121 and the reaction chambers 111. The number of the injection sections 121 and the distributary sections 122 may be flexibly adjusted according to actual requirements and adjusted with consideration to the number of the reaction chambers 111. In the present embodiment, each of the input channels 120 includes two injection sections 121 and three distributary sections 122.

According to the present disclosure, the output unit 13 is used for discharging generated waste liquids by the chemical reaction in the microenvironment building unit 11. The output unit 13 includes two output channels 130, which are disposed between the chemical reaction regions 110, and each of which is in fluid communication with a respective one of the chemical reaction regions 110. Each of the output channels 130 includes a draining section 131 which has a branch shape and which is in fluid communication with corresponding ones of the reaction chambers 111.

In the present embodiment, the alkaline gas that is generated in the microenvironment building unit 11 is ammonia. The ammonia is generated by an acid-base reaction of a sodium hydroxide solution with an ammonia chloride solution after the solutions fluidly flow from the input unit 12 to the microenvironment building unit 11 where the reaction occurs. The acid gas is carbon dioxide. The carbon dioxide is generated by an acid-base reaction of a hydrochloric acid with a sodium carbonate solution after the two fluidly flow from the input unit 12 to the microenvironment building unit 11 where the reaction occurs. The oxygen concentration is controlled, by supplying a cobalt sulfate and a sodium sulfite solution from the input unit 12 to the microenvironment building unit 11, so as to catalyze the sodium sulfite solution with the cobalt sulfate and conduct an oxidation reaction with oxygen in the microenvironment building unit 11. After the acid-base reactions and the oxidation reaction as mentioned above are completed, the generated waste liquids are discharged from the output unit 13.

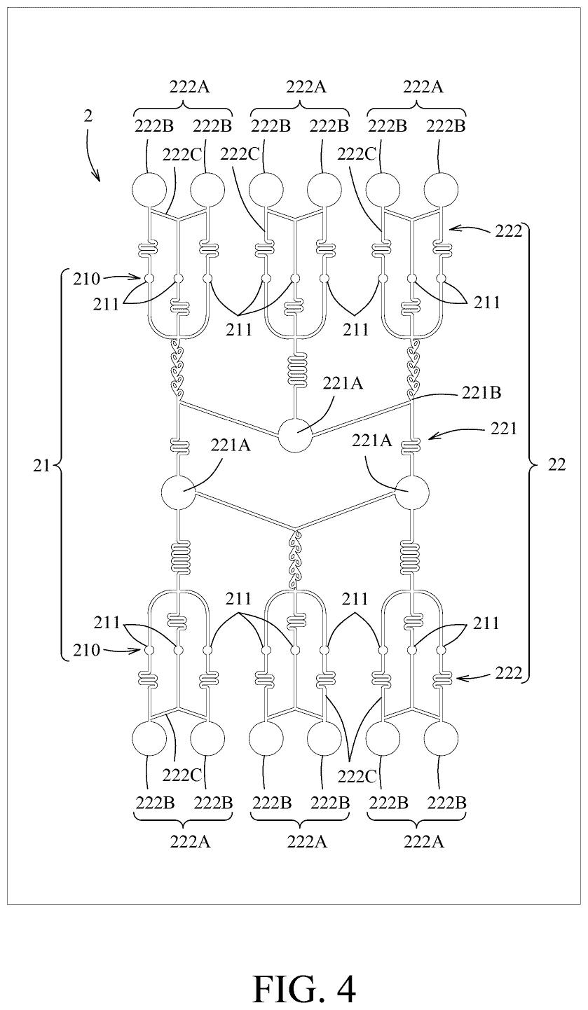

Referring to FIG. 1 and FIG. 4, the drug testing chip 2 includes a testing unit 21 and a channel unit 22 that is in fluid communication with the testing unit 21.

According to the present disclosure, the testing unit 21 may be used for allowing interaction of a therapeutic drug and a cell-containing hydrogel. An orthographic projection of the testing unit 21 falls on the microenvironment building unit 11. The testing unit 21 includes two testing regions 210 which are disposed in opposing directions from each other. Each of the testing regions 210 includes a plurality of testing chambers 211 which are arranged spaced apart from one another. Interaction of the therapeutic drug and the cell-containing hydrogel is conducted in the testing chambers 211. The number of the testing chambers 211 may be flexibly adjusted according to actual requirements. In the present embodiment, each of the testing regions 210 has nine testing chambers 211.

According to the present disclosure, the channel unit 22 includes a first channel portion 221, which is disposed between the testing regions 210 and which is in fluid communication with the testing regions 210, and two second channel portions 222, which are disposed in opposing directions from each other and which are in fluid communication with the testing regions 210. The first channel portion 221 includes a plurality of first ports 221A which are disposed spaced apart from one another, and a hyper-branch communication section 221B which has a hyper-branch shape and which fluidly communicates the first ports 221A and the testing chambers 211. Each of the second channel portions 222 includes a plurality of distributary structures 222A which are in fluid communication with adjacent ones of the testing regions 210. Each of the distributary structures 222A includes two second ports 222B which are spaced apart from each other, and a receiving section 222C which has a branch shape and fluidly communicates the second ports 222B and adjacent ones of the testing chambers 211. The number of the first ports 221A and the distributary structures 222A may be flexibly adjusted according to actual requirements. In the present embodiment, the first channel portion 221 includes three first ports 221A. Each of the second channel portions 222 includes three distributary structures 222A (each of distributary structures 222A is in fluid communication with three testing chambers 211).

According to the present disclosure, the therapeutic drug, through the first ports 221A of the first channel portion 221, is injected and, through the hyper-branch communication section 221B, is diverged and flows into the testing regions 210. Excess therapeutic drug in the testing regions 210 are diverged and flows into the second ports 222B through the receiving sections 222C of the second channel portions 222, and is discharged.

In the present embodiment, since the first channel portion 221 includes three first ports 221A, different types of therapeutic drugs may each be injected from different first ports 221A. In other word, the drug screening platform for the tumor microenvironment simulation of the present disclosure may be injected with three types of therapeutic drugs (that is to say, a first therapeutic drug, a second therapeutic drug, and a third therapeutic drug). The therapeutic drug may include, but is not limit to, Cisplatin, Docetaxel, and Paclitaxel.

Referring to FIG. 4 for further illustration, firstly, the first therapeutic drug may be injected into the first port 221A which is located on the left. The second therapeutic drug may be injected into the first port 221A which is located on the middle. The third therapeutic drug may be injected into the first port 221A which is located on the right. Next, the first therapeutic drug, the second therapeutic drug, and the third therapeutic drug, through the hyper-branch communication section 221B, are diverged and flow into the testing regions 210 and form a first mixture (which is formed by mixing the first therapeutic drug and the second therapeutic drug), a second mixture (which is formed by mixing the second therapeutic drug and the third therapeutic drug), and third mixture (which is formed by mixing the first therapeutic drug and the third therapeutic drug). Then, the therapeutic drugs and the mixtures are each injected into upper nine testing chambers 211 and into lower nine testing chambers 221. Specifically speaking, in the upper nine testing chambers 221, three of the testing chambers 211 which are located relatively on the left accommodate the first mixture; three of the testing chambers 211 which are located relatively in the middle accommodate the second therapeutic drug; three of the testing chambers 211 which are located relatively on the right accommodate the second mixture. In the lower nine testing chambers 221, three of the testing chambers 211 which are located relatively on the left accommodate the first therapeutic drug; three of the testing chambers 211 which are located relatively the middle accommodate the third mixture; three of the testing chambers 211 which are located relatively on the right accommodate the third therapeutic drug.

In other words, the hyper-branch communication section 221B may split flows of the first therapeutic drug, the second therapeutic drug, and the third therapeutic drug into the testing chambers 211 as mentioned above, and simultaneously mix the first therapeutic drug, the second therapeutic drug, and the third therapeutic drug in different combination, thereby splitting flows of the first mixture, the second mixture, and the third mixture into the testing chambers 211 as mentioned above correspondingly.

According to the present disclosure, the cell-containing hydrogel is injected into the second ports 222B of the second channel portions 222 and, through the receiving sections 222C, is diverged and flow into the corresponding testing regions 210. Excess cell-containing hydrogel in the testing regions 210 are diverged and flow into the first ports 221A through the hyper-branch communication section 221B of the first channel portion 221, and is discharged.

According to the present disclosure, the cell-containing hydrogel includes a cell and hydrogel. The type of the cell may include, but is not limit to, a cancer cell, a stromal cell and a fibrocyte. In addition, through the design and cooperation of the testing unit 21 and the channel unit 22, the cell-containing hydrogel injected into the second ports 222B may be a single type of cell-containing hydrogel or a two different types of cell-containing hydrogels.

Specifically speaking, firstly, a first cell-containing hydrogel may be injected into one of the second ports 222B of each of the distributary structures 222A, and a second cell-containing hydrogel may be injected into another one of the second ports 222B of each of the distributary structures 222A. Next, the receiving section 222C of each of the distributary structures 222A may allow the first cell-containing hydrogel and the second cell-containing hydrogel to be diverged and flow into the testing chamber 211, respectively, and allow the first cell-containing hydrogel and the second cell-containing hydrogel to be mixed and flow into a same testing chamber 211.

Referring to FIG. 4 and taking one of the distributary structures 222A for further illustration, the first cell-containing hydrogel, after being injected into the second port 222B which is located on the left, is diverged and flows, through the receiving section 222C, into the testing chambers 211 that are on the left and in the middle. The second cell-containing hydrogel, after being injected into the second port 222B which is located on the right, is diverged and flows, through the receiving section 222C, into the testing chambers 211 that are on the right and in the middle. As a result, the testing chamber 211 which is located on the left accommodates the first cell-containing hydrogel. The testing chamber 211 which is located in the middle, is accommodated with a mixture of the first cell-containing hydrogel and the second cell-containing hydrogel. The testing chamber 211 located on the right accommodates the second cell-containing hydrogel.

It should be noted that the present disclosure is adapted for simultaneously testing three types of therapeutic drugs and two types of cell-containing hydrogels. As a result, the present disclosure may, through performing one test, obtain multiple test results, thereby screening the drugs effectively.

In an operation of the present disclosure, firstly, the cell-containing hydrogel is injected into the second ports 222B so as to split flow of the cell-containing hydrogel into the testing regions 210. Meanwhile, excess cell-containing hydrogel in the testing regions 210 are diverged and flows into the first ports 221A through the hyper-branch communication section 221B of the first channel portion 221, and is discharged. Then, a buffer solution is injected into the first ports 221A, so as to wash the excess cell-containing hydrogel, and is discharged from the second ports 222B. After the cell-containing hydrogel in the testing regions 210 is fixed, an automatic pumping-perfusion-apparatus that is connected externally (not depicted in the Figures) is used to perfuse the therapeutic drug into the first ports 221A so as to split flow of the therapeutic drug into the testing regions 210. A utilization of the automatic-pumping-perfusion-apparatus to perfuse the therapeutic drug simulates a treatment implementation where chemotherapy drugs are injected into a cavum abdominis or cavum thoracis of a human body in a perfusion manner when performing hyperthermic chemoperfusions.

Referring to FIG. 2, the permeable thin film 3 is disposed between the drug testing chip 2 and the tumor microenvironment simulating chip 1. The permeable thin film 3 is used for diffusing the acid gas, the alkaline gas and/or the oxygen concentration generated by the microenvironment building unit 11 to the testing unit 21, thereby simulating the tumor microenvironment that has a proper acid-base-property and/or a different oxygen concentration in the testing unit 21, so as to allow the therapeutic drug and the cell-containing hydrogel interact with each other in a simulated tumor microenvironment. The material of the permeable thin film 3 is not limited, as long as the permeable thin film 3 has a gas permeability and may isolate the microenvironment building unit 11 from the testing unit 21. In the present embodiment, the material of the permeable thin film 3 is polydimethylsiloxane (abbreviated as PDMS).

Referring to FIG. 1, the heating apparatus 4 includes a heating device 41, a temperature-sensing device 42, and a temperature-controlling device 43. The heating device 41 is used for performing the heating treatment on the drug testing chip 2. The temperature-sensing device 42 is used for measuring a temperature of the drug testing chip 2 to generate a signal. The temperature-controlling device 43 is used for receiving the signal generated by the temperature-sensing device 42 to control a temperature of the heating device 41. In order to simulate the treating implementation where a heated chemotherapy drug is injected into cavum abdominis or cavum thoracis of a human body as performing the hyperthermic chemoperfusions, a heating treatment is performed on the drug testing chip 2 by using the heating apparatus 4, so that a warm tumor microenvironment in the testing unit 21 of the drug testing chip 2 is simulated, thereby allowing the therapeutic drug and the cell-containing hydrogel to interact with each other in the simulated tumor microenvironment.

In summary, in the drug screening platform for the tumor microenvironment simulation in the present disclosure, the microenvironment building unit 11 may allow performing chemical reactions (an acid-base reaction and an oxidation reaction) to generate the acid gas, the alkaline gas and/or the oxygen concentration for simulating the tumor microenvironment, and then, through the permeable thin film 3, the acid gas, the alkaline base and/or the oxygen concentration may diffuse to the testing unit 21 of the drug testing chip 2. Then, optionally through the heating apparatus 4, a warm tumor microenvironment in the testing unit 21 may be simulated so that the therapeutic drug and the cell-containing hydrogel in the testing unit 21 may interact with each other in the simulated tumor microenvironment, thereby obtaining a testing result that is both accurate and correct. In addition, through the design and cooperation of the testing unit 21 and the channel unit 22, the drug screening platform for the tumor microenvironment simulation of the present disclosure may optionally test three types of therapeutic drugs and two types of cell-containing hydrogels, thereby obtaining eighteen testing results that is both accurate and correct by performing only one test.

However, the above are merely the embodiments of the present invention, and should not be used to limit the scope of the present disclosure. Any simple equivalent variations and modifications made according to the claims and the content of the specification of the present disclosure should fall within the scope covered by the present disclosure.

What is claimed is:

1. A drug screening platform for tumor microenvironment simulation, comprising:
a tumor microenvironment simulating chip, including a microenvironment building unit which is used for conducting a chemical reaction to generate an acid gas, an alkaline gas and/or an oxygen concentration for simulating the tumor microenvironment;
a drug testing chip, including a testing unit which is used for allowing interaction of a therapeutic drug with a cell-containing hydrogel, an orthographic projection of said testing unit falling on said microenvironment building unit; and
a permeable thin film disposed between said tumor microenvironment simulating chip and said drug testing chip, and used for diffusing the acid gas, the alkaline gas and/or the oxygen concentration generated by said microenvironment building unit to said testing unit.

2. The drug screening platform for the tumor microenvironment simulation as claimed in claim 1, wherein said microenvironment building unit has two chemical reaction regions which are disposed in opposing directions from each other, each of said chemical reaction regions having a plurality of reaction chambers which are arranged spaced apart from one another, the chemical reaction being conducted in said plurality of reaction chambers.

3. The drug screening platform for the tumor microenvironment simulation as claimed in claim 2, wherein said tumor microenvironment simulating chip further includes an input unit and an output unit, which are in fluid communication with said microenvironment building unit.

4. The drug screening platform for the tumor microenvironment simulation as claimed in claim 3, wherein said input unit includes two input channels which are disposed in opposing directions from each other and each of which is in fluid communication with a respective one of said chemical reaction regions, each of said input channels including a plurality of injection sections and a plurality of distributary sections which fluidly communicate said plurality of injection sections and said plurality of reaction chambers.

5. The drug screening platform for the tumor microenvironment simulation as claimed in claim 3, wherein said output unit includes two output channels, which are disposed between said chemical reaction regions, and each of which is in fluid communication with a respective one of said chemical reaction regions, each of said output channels including a draining section which has a branch shape and which is in fluid communication with corresponding ones of said plurality of reaction chambers.

6. The drug screening platform for the tumor microenvironment simulation as claimed in claim 1, wherein said testing unit includes two testing regions which are disposed in opposing directions from each other, each of said testing regions including a plurality of testing chambers which are arranged spaced apart from one another, the interaction of the therapeutic drug and the cell-containing hydrogel being conducted in said plurality of testing chambers.

7. The drug screening platform for the tumor microenvironment simulation as claimed in claim 6, wherein said drug testing chip further includes a channel unit that is in fluid communication with said testing unit.

8. The drug screening platform for the tumor microenvironment simulation as claimed in claim 7, wherein said channel unit includes
a first channel portion which is disposed between said testing regions and which is in fluid communication with said testing regions, said first channel portion including a plurality of first ports which are disposed spaced apart from one another and a hyper-branch communication section which has a hyper-branch shape and which fluidly communicates said plurality of first ports and said plurality of testing chambers, and
two second channel portions, which are disposed in opposing directions from each other and which are in fluid communication with said testing regions, each of said second channel portions including a plurality of distributary structures which are in fluid communication with adjacent ones of said testing regions, each of said plurality of distributary structures including two second ports which are spaced apart from each other, and a receiving section which has a branch shape and fluidly communicates said second ports and adjacent ones of said plurality of testing chambers.

9. The drug screening platform for the tumor microenvironment simulation as claimed in claim 1, further comprising a heating apparatus for performing a heating treatment on said drug testing chip.

10. The drug screening platform for the tumor microenvironment simulation as claimed in claim 9, wherein said heating apparatus includes a heating device, a temperature-sensing device, and a temperature-controlling device, said heating device being used for performing the heating treatment on said drug testing chip, said temperature-sensing device being used for measuring a temperature of said drug testing chip to generate a signal, said temperature-controlling device being used for receiving the signal generated by said temperature-sensing device to control a temperature of said heating device
